(19) United States
(12) Patent Application Publication
(10) Pub. No.: US 2025/0032686 A1
(43) Pub. Date: Jan. 30, 2025

(54) MEDICAL TREATMENT APPARATUS WITH WEAR MONITORING OF ITS NEGATIVE PRESSURE SOURCE

(71) Applicant: Fresenius Medical Care Deutschland GmbH, Bad Homburg (DE)

(72) Inventors: Matthias Baecker, Bad Homburg (DE); Lena Goetz, Bad Homburg (DE); Stefan Mueller, Bad Homburg (DE); Martin Thys, Bad Homburg (DE); Eva Warmuth, Bad Homburg (DE)

(21) Appl. No.: 18/551,443

(22) PCT Filed: Mar. 23, 2022
(86) PCT No.: PCT/EP2022/057609

§ 371 Date: Sep. 20, 2023

(30) Foreign Application Priority Data
Mar. 24, 2021 (DE) 10 2021 107 396.0

(51) Int. Cl. A61M 1/16

(52) U.S. Cl. CPC A61M 1/1601; A61M 2205/3331

(57) ABSTRACT

The present disclosure relates to a medical treatment apparatus, which respectively includes, or is respectively connected to, a vacuum line, a negative pressure source, a pressure measuring device, a determining device, an output device, a storage device, a reading device, and at least one control device or closed-loop control device. By using the stated devices, the condition of the negative pressure source is to be measured based on the measured pressure reference profile. The present disclosure further relates to control device or closed-loop control device as well as to a digital storage device, a computer program product, and a computer program.

### CROSS-REFERENCE TO RELATED APPLICATIONS

The present application is the national stage entry of International Application No. PCT/EP2022/057609, filed on Mar. 23, 2022, and claims priority to Application No. DE 102021107396.0, filed in the Federal Republic of Germany on Mar. 24, 2021, the disclosures of which are expressly incorporated herein in their entirety.

### TECHNICAL FIELD

The present disclosure relates to a medical treatment apparatus as described herein, to a control device or closed-loop control device as described herein, to a digital storage medium as described herein, to a computer program product as described herein, and to a computer program as described herein.

### BACKGROUND

Medical treatment apparatuses, whose operation requires at least occasionally the use of compressed air below atmospheric pressure, are known. Among such medical treatment apparatuses are for example some blood treatment apparatuses which are used in particular for hemodialysis, hemofiltration and hemodiafiltration. They have one thing in common which is that during the extracorporeal blood treatment executed by using them, the blood to be treated flows along an extracorporeal blood circuit and through a blood treatment unit. In the apparatuses for hemodialysis, hemofiltration and hemodiafiltration, the blood treatment unit is a dialyzer or filter which, in simple terms, is separated into a blood chamber and a dialysis liquid chamber by a semipermeable membrane. During the blood treatment by hemodialysis or hemodiafiltration, the blood flows through the blood chamber, while a dialysis liquid flows through the dialysis liquid chamber.

The conveyance accuracy of the negative pressure source used to generate negative pressure, usually a compressor, may change over time and duration of its use, and the negative pressure source may ultimately even fail or malfunction or break down. Causes or reasons behind changes to be observed may include leaks, swelling of materials, deposits inside the conveying device and/or the lines, and wear. While leaks may be reliably detected in a simple manner performing an automatic integrity test, for example a pressure holding test, the extent of the wear that occurs over time is not easily recognizable or detectable with the known tests for leaks.

In order not to endanger the treatment of the patient and their safety, negative pressure sources provided for this purpose are designed so robustly that operational wear is as low as possible during their service life. However, the ability or capacity of the negative pressure source to build up the negative pressure may change over time.

### SUMMARY

It is an aspect of the present disclosure to describe a medical treatment apparatus (in short: treatment apparatus) having a negative pressure source. Furthermore, this disclosure describes a control device or closed-loop control device (in short: control device, which may optionally also regulate) with which steps for checking the negative pressure source may be prompted. In addition, a digital storage medium, a computer program product and a computer program are described herein.

Thus, according to the present disclosure, a medical treatment apparatus is described which respectively includes, or is respectively connected to, at least one vacuum line, a negative pressure source, a pressure measuring device, a determining device, an output device, a storage device, a reading device, and at least one control device or closed-loop control device.

The negative pressure source, which is for example a compressor, is in fluid communication or conveyance communication with the vacuum line in order to be able to build up or change a negative pressure there.

By using the pressure measuring device, pressure values are measured in the vacuum line, which pressure values serve to achieve a pressure value profile over time. A pressure value profile is herein at least a first pressure value and a second pressure value (or more than two pressure values), which respectively prevail in the vacuum line at a first point in time and at a second point in time different from the first point in time, are measured, or could be measured, individually, in groups or all together.

The determining device is configured to determine at least one value to be assessed from the pressure value profile measured by the pressure measuring device or from the pressure value profile generated by the negative pressure source.

The output device is configured to output values and/or signals, e.g., alarms.

At least one reference pressure profile or at least one reference value related to, or obtained from, a reference pressure profile is stored or kept in the storage device.

Alternatively or additionally, there is stored in the storage device at least one measurement which is assigned to the value to be assessed of the pressure value profile and which indicates present/actual wear of the negative pressure source.

The reading device is configured to read out from the storage device the at least one stored reference pressure profile, the at least one reference value, or the assigned measurement.

The control device or closed-loop control device is configured to generate, build up, or change negative pressure in the vacuum line using the negative pressure source. It is further configured to measure a negative pressure prevailing in the vacuum line by using the pressure measuring device in order to determine a measured pressure value profile.

Furthermore, the control device or closed-loop control device is configured to determine using the determining device, a value to be assessed from the pressure value profile measured by the pressure measuring device. The control device or closed-loop control device may in some embodiments also be configured to output this value.

The control device or closed-loop control device is further configured to read out using the reading device the at least one stored reference pressure profile, the at least one reference value (corresponding to the value to be assessed) from this reference pressure profile or a measurement from the storage device, which is assigned to the value to be assessed.

Furthermore, the control device or closed-loop control device is configured to output a result, being determined by the control device or closed-loop control device, of a comparison between the read reference value on the one hand and the value to be assessed on the other hand or the assigned measurement, wherein said output is carried out by the output device.

When “programmed” or “configured” is mentioned here, it is also disclosed that these terms may be interchanged with each other.

Determining, in particular of data or values, may herein be or encompass examining an existence or non-existence, obtaining, detecting/capturing, measuring, evaluating, processing, comparing, assessing/appraising, estimating, concluding, calculating, achieving, attaining, reaching and/or recognizing.

According to the present disclosure, there is further proposed a control device or closed-loop control device as defined herein.

All, several, or some of the steps disclosed herein may be prompted or executed by the control device or closed-loop control device of the medical treatment apparatus.

A control device or closed-loop control device in particular a control device or closed-loop control device according to the present disclosure, may be included by or connected to the medical treatment apparatus according to the present disclosure. The control device or closed-loop control device is configured to prompt, execute control and/or regulate—in particular automatically—the methods disclosed herein by interacting with further devices or apparatuses of the medical treatment apparatus, in particular as disclosed herein.

An interaction may be, or may encompass actuation, control or regulation. An interaction may be or may require a signal communication.

The medical treatment apparatus according to the present disclosure may, for each of the steps mentioned herein, have, or be connected to, a correspondingly suitable and/or configured device or apparatus, such as an evaluation unit for evaluation, a pressure measuring device for pressure measurement, etc.

A storage medium according to the present disclosure (here also referred to as carrier), in particular digital, in particular non-volatile, in particular in the form of a diskette, RAM, ROM, CD, hard disc, DVD, USB stick, Flashcard, SD card, EPROM or NOVRAM, particularly with electronically or optically readable control signals, may be configured such that to configure a control device or closed-loop control device into a control device or closed-loop control device according to the present invention.

Alternatively or additionally, the digital storage medium may be configured in order to configure a medical treatment apparatus into a medical treatment apparatus according to the present disclosure.

A computer program product according to the present disclosure includes a volatile, transient program code or one stored on a machine-readable carrier. By which computer a control device or closed-loop control device is configured into a control device or closed-loop control device according to the present disclosure.

Alternatively or additionally, a medical treatment apparatus may be configured into a medical treatment apparatus according to the present disclosure by the computer program product.

The term “machine readable carrier” as used herein, refers in certain embodiments of the present disclosure to a carrier, which contains data or information interpretable by software and/or hardware. The carrier may be a data carrier, such as a diskette, a CD, a DVD, a USB stick, a flashcard, an SD card, an EPROM or the like.

A computer program according to the present disclosure includes a program code, by which a control device or closed-loop control device is configured into a control device or closed-loop control device according to the present disclosure, or a medical treatment apparatus is configured into a medical treatment apparatus according to the present disclosure.

According to the present disclosure, a computer program product may be understood as a computer program stored for example on a carrier, an embedded system being a comprehensive system with a computer program (e.g., electronic device with a computer program), a network of computer implemented computer programs (e.g., client/server-system, a cloud computing system, etc.), or a computer on which a computer program is loaded, runs, is stored, is executed or developed.

According to the present disclosure, a computer program may be understood to mean, for example, a physical, marketable software product which includes a program.

In all of the aforementioned and the following statements, the use of the expression “may be” or “may have” and so on, is to be understood synonymously with the expression “preferably is” or “preferably has,” and so on respectively, and is intended to illustrate an embodiment according to the present disclosure.

Whenever numerical words are mentioned herein, the person skilled in the art shall recognize or understand them as indications of numerical lower limits. Unless it leads the person skilled in the art to an evident contradiction, the person skilled in the art shall comprehend the specification for example of “one” (also: “a/an”) as encompassing “at least one”. This understanding is also equally encompassed by the present disclosure as the interpretation that a numeric word, for example, “one” (also: “a/an”) may alternatively mean “exactly one”, wherever this is evidently technically possible for the person skilled in the art. Both understandings are encompassed by the present disclosure and apply herein to all used numerical words.

When an embodiment is mentioned herein, it is then an exemplary embodiment according to the present disclosure.

When it is disclosed herein that the subject-matter according to the present disclosure includes one or several features in a certain embodiment, it is also respectively disclosed herein that the subject-matter according to the present disclosure does, in other embodiments, likewise according to the present disclosure, explicitly not include this or these features, for example, in the sense of a disclaimer. Therefore, for every embodiment mentioned herein it applies that the converse embodiment, e.g., formulated as negation, is also disclosed.

Embodiment according to the present disclosure may include one or several of the aforementioned or following features in any technically possible combination.

In some embodiments, the medical treatment apparatus according to the present disclosure includes, or is connected to, an evaluation device. The evaluation device is configured to assign a measure of wear to the result of the comparison between the read reference value, on the one hand, and the value to be assessed, on the other hand. In these embodiments, the control device or closed-loop control device is further configured for prompting the evaluation device to assign a measure of wear to the result of the comparison between the read reference value on the one hand and the value to be assessed on the other hand, by using the evaluation device. A measure determined in this way may in turn be output by the output device.

In several embodiments of the medical treatment apparatus according to the present disclosure, the at least one reference value and/or the value to be assessed is a time value read or measured from the pressure value profile, for example the time that elapses until a predetermined negative pressure value is reached, e.g., the lower negative pressure threshold. Also, alternatively, the reference value is a mathematical parameter of the pressure value profile, for example an angle determined or determinable from the pressure value profile, a slope/gradient of the profile in a diagram, etc.

In some embodiments, the control device or closed-loop control device is configured to determine whether the corresponding value from the measured pressure value profile, the result of the comparison, and/or the measure of wear is within predetermined limits, exceeds or falls below a limit value, exceeds a minimum value, and/or does not exceed a maximum value, or the evaluation or comparison consists thereof. Hereby, an evaluation is done, e.g., on the basis of a criterion (limit value, range, maximum value, etc.). As a criterion, for example for the output of an alarm, it may be evaluated, for example, whether an angle is smaller than a predetermined angular degree and/or whether the time required to build up negative pressure exceeds a predetermined duration and/or the like.

In several embodiments, the result of the evaluation or comparison, is or encompasses a quantitative statement, for example, a statement in percent, for instance relative to the reference value.

In some embodiments, the control device or closed-loop control device is configured to output an acoustic and/or optical alarm using the output device. The alarm is output, for example, if the evaluation leads to results that have been defined in advance as inadmissible, in particular when predetermined limits are exceeded, in particular when the value falls below the lower limit value or exceeds the upper limit value, when a permissible range of values is left, or when a predetermined absolute value is exceeded. These results defined as inadmissible may be stored, for example in the storage device mentioned herein.

In several embodiments, the medical treatment apparatus includes or is connected to a storage device in which the control device or closed-loop control device stores or is programmed to store the result of the evaluation or comparison, namely in the storage device referred to herein, for the reference value by way of example.

In some embodiments, it may be provided that the previously measured pressure value profiles are also taken into account during the evaluation by the evaluation device or that they are included in the determination of the measure of wear.

This storage device may be part of the medical treatment device, for example in a section that is regularly read out during maintenance activities. However, it may also be arranged remotely from the medical treatment apparatus, e.g., at a site assigned to or included in the maintenance or monitoring of the treatment apparatus.

In some embodiments, the control device or closed-loop control device is configured to determine, e.g., calculate, a wear curve based on the time for the negative pressure source from the stored results or measurements. Alternatively, it is configured to prompt this determining.

For this purpose, various pressure profiles may be evaluated, for example, in order to determine and optionally quantify a trend at an early stage—i.e., developments that become apparent in the medium or long term—e.g., on the basis of their slope/gradient or other properties.

In several embodiments, the stored curves or a wear curve based thereon may, if needed, be read out for example by a service technician. Alternatively, the data memory may be provided on an external device. Thus, results can be fed into e.g., a network and stored there. If necessary, the results may then be retrieved from multiple devices.

In some embodiments, a probability of occurrence of a malfunction due to wear of the negative pressure source is determined according to known methods and communicated, e.g., by storage in the readable data memory, by display, by alarm, etc. In addition or alternatively, the point of time or the time period at which such a malfunction is likely to occur may be determined and communicated.

In several embodiments, known methods of trend analysis are used to determine, among other things, the probability of occurrence of a malfunction due to wear of the negative pressure source or the point of time or the time period of said occurrence.

The hardware or software required for carrying out the aforementioned determining of probability, time/period, and/or the presentation and/or transmission of the results and the like is optionally provided and accordingly set up, configured, and/or programmed.

Known methods of trend analysis encompass simple averaging, determination of moving averages, determination of least square deviation, first-order exponential smoothing, etc.

In several embodiment, the medical treatment apparatus is embodied as a blood treatment apparatus, in particular as a hemodialysis apparatus, a hemofiltration apparatus, hemodiafiltration or as an apparatus for performing a separation procedure.

The reference value or reference pressure profile as disclosed herein may be, or may have been, stored in the storage device for example by the manufacturer during production or by the technician during commissioning, which storage device in turn may be part of the negative pressure source or the medical treatment apparatus including the negative pressure source. The invariable reference value or the reference pressure profile may be read out from it when the steps mentioned herein are being carried out.

Reference values may be stored for example in a Non-Volatile Random Access Memory (NOVRAM); a NOVRAM is a memory module which, in principle, may be written to cyclically, but in which the last valid data content is nevertheless saved in the internal ROM area in the event of an external power failure. A capacitor integrated in a NOVRAM supplies the energy required to copy the contents of the RAM section, which is also integrated in the NOVRAM, to the ROM section in the event of a failure of the externally applied voltage.

In several embodiments, if a gradual or increasing wear of the negative pressure source is detected over a plurality of (at least two) pressure profiles at different points in time, an alarm or notification may be issued in this regard.

For example, the medical treatment apparatus may issue a warning or indication that the negative pressure source is due for maintenance, repair or replacement because of wear. In this way, replacement may be carried out in good time, i.e., before a complete failure actually occurs, which in turn may advantageously help to reduce the downtime of the treatment apparatus. Maintenance or repair of the negative pressure source may be scheduled at an early stage and thus, unlike maintenance carried out as a reaction, cannot be carried out at an inopportune time, i.e., at a possibly unsuitable time.

Since a worn negative pressure source may be identified according to the present disclosure without a dedicated test already during the generation of negative pressure regularly required for the ordinary operation of the medical treatment apparatus, the time at which maintenance or repair of the negative pressure source should best be tackled, may be detected comparatively early. In several embodiments, the results of the evaluation (as evaluation results, degree of wear, etc.) are displayed on, or by, the output device, e.g., on the treatment apparatus (display) or on an external monitor, display, or the like, or they are printed (out).

In several embodiments, the results of the evaluation (as evaluation results, degree of wear, etc.) are displayed or printed on, or using, the output device, e.g., on the treatment apparatus (display) or on an external monitor, display, or the like.

In some embodiments of the medical apparatus according to the present disclosure, the control device is further configured to prevent a treatment option performed by the medical apparatus and/or to stop a pump of the medical apparatus, preferably a pump that conveys medical liquid, in particular dialysis liquid. According to the present disclosure, this is done when the evaluation results (as evaluation results, degree of wear, etc.) indicate that the wear of the negative pressure source is too advanced.

When reference is made herein to measuring a pressure, in several embodiments this term may also extend to determining the pressure sought, for example by calculating the pressure from provided values.

When a signal connection or communication connection between two components is mentioned herein, this may be understood to mean a connection that is present during use. Likewise, it may be understood to mean that there is a preparation for such a signal connection (in a wired, wireless or in another manner) for example by coupling the two components, for example by pairing, etc.

Pairing is understood as a process that takes place in connection with computer networks to establish an initial link or connection between computer units for the purpose of communication. The best known example of this is the establishing of a Bluetooth connection, by which various devices (e.g., smartphone, headphones) are connected with each other. Pairing is sometimes also referred to as bonding.

In some embodiments, the value to be assessed is not or does not include a torque or a pump speed.

In several embodiments, the negative pressure source is not a piston pump and/or is not a centrifugal pump.

In some embodiments, the negative pressure source is not a liquid pump or is not used to pump liquids.

In several embodiments, the negative pressure source is not a central vacuum facility, such as of a hospital. The negative pressure source is preferably part of the medical treatment apparatus, e.g., encompassed by its housing.

In some embodiments, the suction side of the compressor referred to herein is used as the negative pressure source. Likewise, the pressure side of the compressor may be used as the negative pressure source, for example, by appropriate guiding of the compressed fluid, use of a venturi nozzle, and/or similar.

In several embodiments, the control device is not programmed to determine a deflection of a piston of a pump using piston deflection sensors in order to determine wear.

By some embodiments according to the present disclosure, one or several of the advantages mentioned herein may be achievable, which include the following:

The present devices, systems, and methods render possible to state a reliable fact about the functional efficiency and, in particular, about the degree of wear of the negative pressure source.

Another advantage of the present devices, systems, and methods may be that a medical treatment apparatus is enabled to automatically check its negative pressure source. The effort of a qualified service technician or user is not required for said check, which may help to save costs.

In addition, deviations in the conveying activity of a negative pressure source that occur for the first time between visits by the qualified service technician or checks by the user may be detected at an early stage, for example as part of a routine daily check or use of the medical treatment apparatus during treatment.

Another advantage may be the improvement of the user-friendliness of a generic treatment apparatus. According to the present disclosure, the user is not burdened with checking the negative pressure source. Downtime of the treatment apparatus during use is minimized, if not eliminated, by the present invention.

Detecting the need to replace the negative pressure source in time may thus increase the reliability of the medical treatment apparatus, and hence the safer for the patient it may operate.

Finally, it may be advantageous that by the present devices, systems, and methods, the statistical evaluation of failure figures relating to the negative pressure source may be improved. This advantageously helps to identify real errors and to reduce the number of unreported cases of worn negative pressure sources or to eliminate them completely.

### BRIEF DESCRIPTION OF THE DRAWINGS

In the following, the present devices, systems, and methods are described on the basis of preferred embodiments thereof with reference to the attached drawings. The medical treatment apparatus according to the present disclosure is described using the example of a hemodialysis apparatus. However, the devices, systems, and methods may also be used in the same way for other treatment apparatuses, for example for a hemodiafiltration apparatus. In the figures, the following applies:

FIG. 1 shows a highly simplified representation of a flow diagram of a medical treatment apparatus in a first embodiment.

FIG. 2 shows two exemplary pressure value profiles in a pressure-time-diagram during the building-up of a negative pressure and the output of a measure of the wear of the negative pressure source which respectively builds up the negative pressure.

FIG. 3 shows a quantitative assessment of the wear condition of a negative pressure source in a further pressure-time-diagram.

FIG. 4 shows a highly simplified representation of a flow diagram of a further component of a medical treatment apparatus in an exemplary embodiment.

### DETAILED DESCRIPTION

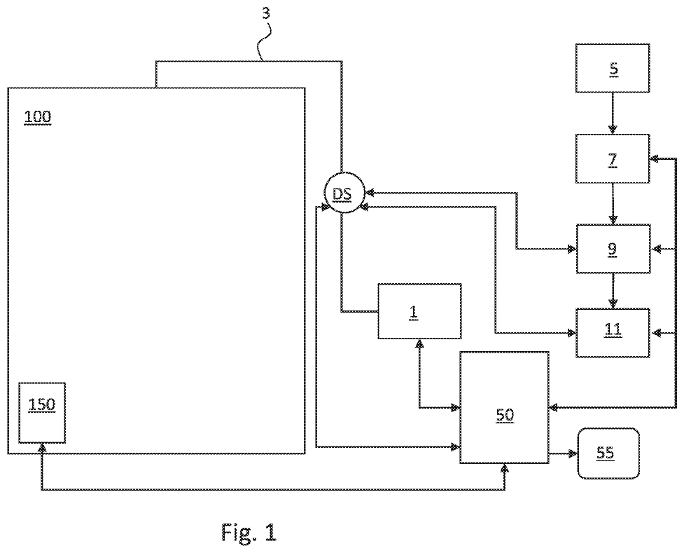

FIG. 1 shows a highly simplified representation of a flow diagram or course diagram of a medical treatment apparatus 100 in a first embodiment.

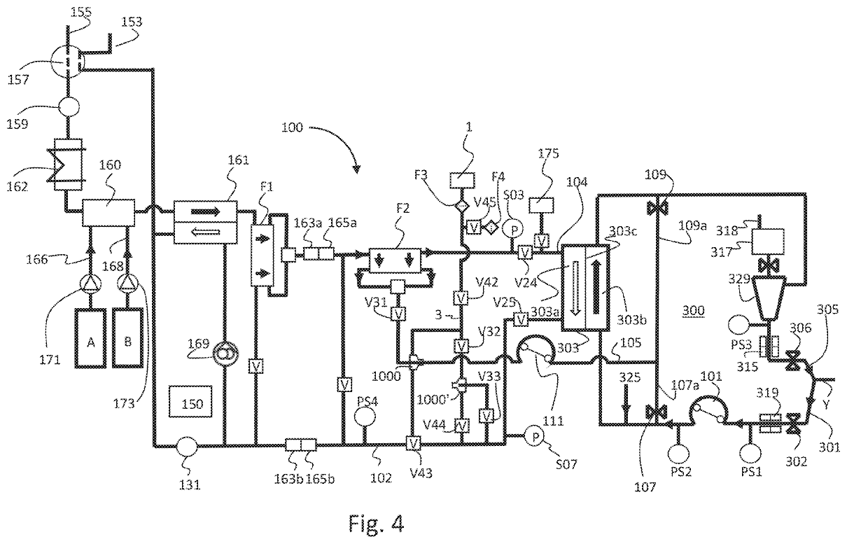

The major part of the medical treatment apparatus 100 is represented as a “black box”. It includes both the blood side with its extracorporeal blood circuit and the hydraulic side with a wide variety of lines, pumps, valves and the like. A blood filter, via which the blood side and the hydraulic side are in communication, is also encompassed by the “black box”. For details of a possible design of the medical treatment apparatus 100, reference is made to FIG. 4.

The medical treatment apparatus 100 is, via a vacuum line 3, in fluid communication or in conveying communication with a negative pressure source 1 for the purpose of building up or changing a negative pressure in the vacuum line 3 prompted by a control device or closed-loop control device 50.

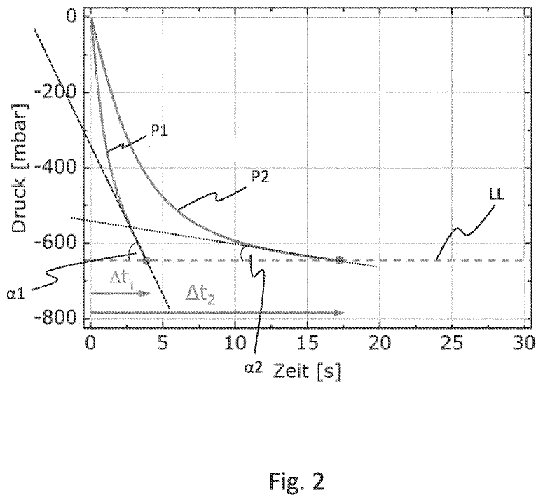

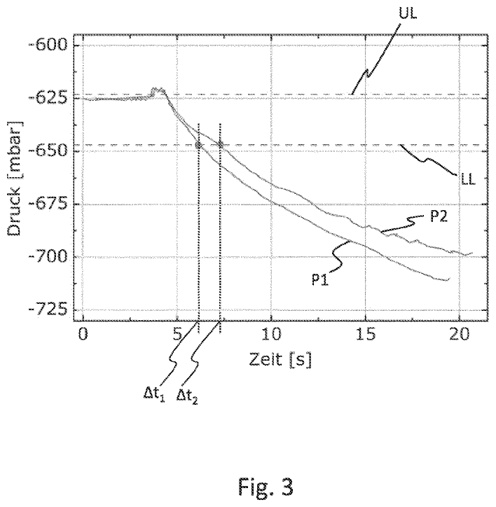

A pressure measuring device DS is arranged on or in the vacuum line 3 for measuring the pressure prevailing within the vacuum line 3. By continuous, or multiple, pressure measurement during the building-up of the negative pressure or the change of an already given negative pressure, a pressure value profile Px (see the pressure value profiles P1, P2 of the following figures) may be determined. A pressure value profile Px may thus herein be understood as a series of pressure values that can be combined to form a continuous profile, for example by interpolation and extrapolation, which profile could be represented graphically as a curve or line, as was done for the pressure value profiles P1, P2 of FIG. 2 and FIG. 3.

A determining device 9, which is also provided, serves to determine at least one value to be assessed Δt₁, α1, Δt₂, α₂, etc., from the pressure value profile P1, P2 measured by the pressure measuring device DS.

If the value to be assessed is, for example, an indication of how long the negative pressure source 1 requires from the time at which the building-up of a predetermined negative pressure using the negative pressure source 1 is started until the predetermined negative pressure is reached, the determining device 9 determines a value Δt₁ for the measured pressure value profile P1 or the value Δt₂ for the measured pressure value profile P2.

If, on the other hand, the value to be assessed is an angle which results from the geometric course of the measured pressure value profile P1 or P2, then the angle α1, for example of FIG. 2 or the corresponding angle α1 for the pressure value profile P1, or based on it, is determined, or respectively the angle α2 is determined for the pressure value profile P2 as the value to be assessed, wherein said determining is done by using the determining device 9.

For details on determining and assessing the value to be assessed, reference is made to the following FIGS. 2 and 3 and their description.

In a storage device 5, there is stored optionally a) at least one reference pressure profile, from which a reference value may be determined which is suitable with respect to the value to be assessed Δt₁, α1, Δt₂, α2, . . . of the pressure value profile P1, P2, b) a reference value—mostly already based on a reference pressure profile or obtained therefrom—for the value to be assessed Δt₁, α1, Δt₂, α2, . . . and/or c) at least one measure (here exemplarily indicated in “%”), which is respectively assigned to the value to be assessed Δt₁, α1, Δt₂, α2, . . . of the pressure value profile P1, P2. The measure indicates a wear of the measured negative pressure source 1 present at the time of measuring the pressure value profile P1, P2 in dependence on the value or amount of the value to be assessed Δt₁, α1, Δt₂, α2, . . . . The contents according to a), b) and c) may be stored in the storage device 5 as alternatives to each other or in addition to each other.

The reference value may thus be or encompass, for example, a time required to build up a predetermined negative pressure, an angular indication of the angle between the reference pressure profile and, for example, a lower control limit LL (see FIG. 2), a horizontal or other reference line in a pressure-time-diagram, or the like.

The reference pressure profile, which is not shown in the figures, and correspondingly the reference value, which was obtained or can be obtained from the reference pressure profile, serve in some embodiments as a comparison value for the value or the characteristic of the value to be assessed, which was determined by the determining device 9 and which is shown in FIG. 2 as Δt₁ or α1 for the pressure value profile P1 or as Δt₂, α2 for the pressure value profile P2.

The reference pressure profile may, for example, be a pressure value profile recorded or collected at a negative pressure source 1 in its new condition. In this case, the pressure value profile P1 shown in FIG. 2 and FIG. 3 could serve as the reference pressure profile.

A reading device 7 is provided and configured for reading out values from the storage device 5, in particular the at least one reference value stored therein, the reference pressure profile stored therein in order to determine a reference value based on it, or the measure of wear stored therein which may be stored in the storage device 5 for different characteristics or values of the value to be assessed, being respectively assigned thereto.

An evaluation device 11 is optionally provided and configured for assigning a measure of wear with a result that may be achieved when the value to be assessed is related to or compared to the reference value.

The control device or closed-loop control device 50 may correspond to a herein outlined control device or closed-loop control device 150 of the medical treatment apparatus 100 or may be provided to be separate therefrom, but being at least in signal connection thereto.

The control device or closed-loop control device 50 is further configured to cause the pressure measuring device DS to measure a negative pressure prevailing in the vacuum line 3, whereby the measured pressure value profile P1, P2 is determined.

Furthermore, the control device or closed-loop control device 50 is configured to enable reading out the at least one stored reference value by the reading device 7, reading out the reference value from the stored reference pressure profile or reading out the measure assigned to the value to be assessed. Said reading out is respectively done from the storage device 5.

Finally, the control device or closed-loop control device 50 is configured to prompt the determining device 9 to determine the value to be assessed from the measured pressure value profile P1, P2.

For assigning a measure of wear to a result that can be obtained when the value to be assessed is related or compared to the reference value, the control device or closed-loop control device 50 acts accordingly on the evaluation device 11 provided for this purpose.

The control device or closed-loop control device 50 is also configured to prompt/effect outputting a result of the comparison or the measure of wear. It can make use of an output device 55 for this purpose.

In some embodiments, the output device 55 is part of the medical treatment apparatus 100, as is optionally any other device shown in FIG. 1. In other embodiments, it is separate therefrom. The output device 55 and the treatment apparatus 100 are preferably in signal communication, which again may optionally be true for any other device shown in FIG. 1.

The control device or closed-loop control device 50 is configured to have an acoustic and/or visual, haptic or other alarm output by the output device 55, should the evaluation lead to results which have been defined in advance as an alarm event or as alarming. Such results may in particular be those that lie outside predetermined limits, in particular fall below a lower limit value or exceed an upper limit value, leave a permissible value range and/or exceed a predetermined amount.

The arrows in FIG. 1 represent, without being limited thereto, signal communications, in particular data communications, which may be implemented wired or wirelessly.

In FIG. 1, the devices described supra are shown next to the medical treatment apparatus 100. It may be encompassed by the present disclosure that one, some or all of these devices are integrated in the medical treatment apparatus 100, or at least in the housing thereof.

FIG. 2 exemplarily shows the two pressure value profiles P1, P2 already mentioned above in a pressure-time-diagram, which were measured during the building-up of negative pressure in the vacuum line 3. Reference is also made in this regard to the description of FIG. 1.

For each of the measured pressure value profiles P1, P2, one or more values may be determined, which need to be assessed with the aim of learning about the wear condition of the negative pressure source 1.

As explained above, the value to be assessed may be, for example, an angle or a time duration for which reference values (as angles or time durations) may be stored in the storage device 5, or for which suitable reference values may be read or measured or differently determined from an optionally stored reference pressure profile (in this case, therefore, again an angle or a time duration).

A comparison of the value to be assessed with the corresponding reference value may, when a suitable evaluation is chosen, may render possible stating a fact about the wear of the negative pressure source 1.

Not only the pressure value profiles P1, P2 may have been measured using the pressure measuring device DS, but in some embodiments, this also applies to the reference pressure profile. The latter may have been measured by the pressure measuring device DS, e.g. in connection with an initial commissioning of the medical treatment apparatus 100 and/or of the negative pressure source 1, or after completion of maintenance thereof, and stored in the storage device 5.

In the example of FIG. 2, for example Δt₁ corresponds to the time required by a new negative pressure source 1 to build up the negative pressure, which is why the pressure profile P1 is considered in the following to be the reference pressure profile, while Δt₂ is the time required by a negative pressure source 1 to build up the negative pressure, wherein said negative pressure source 1 already shows signs of wear due to its repeated use.

If, for example, the time Δt₂ required to build up the negative pressure in the vacuum line 3 by the negative pressure source 1 which is to be checked is measured, a qualitative and/or quantitative value for indicating the current state of wear of the negative pressure source 1 may be determined from this by comparison with the time Δt₁, which is discussed here as an example of a reference value. The shorter the time Δt₂ required to build up negative pressure, the better the condition of the negative pressure source 1. The greater the difference between Δt₁ and Δt₂, the greater the wear may be assumed herein.

FIG. 2 shows that the angle α1 at which the pressure value profile P1 intersects the lower control limit LL, which is explained in FIG. 3, is considerably larger than the angle α2 at which the pressure value profile P2 intersects the lower control limit LL (α1>α2). According to the present disclosure, an angle, e.g. the angle α2, may therefore also be considered as the value to be assessed.

In the example of FIG. 2, for example, the angle α1 corresponds to the angle at which the pressure value profile P1 of a negative pressure source 1 with a first capacity (for example, the capacity of a new negative pressure source) intersects the lower control limit LL (or another reference line or reference straight line), while the angle α2 is the angle at which the pressure value profile P2 of a negative pressure source 1 with a second capacity being different from the first (for example, the capacity of a negative pressure source that has been in use for a certain time) intersects this lower control limit LL.

As explained for FIG. 1, different time durations Δtₓ or angles αx may be stored in the storage device 5 for an alternative assessment of the wear of a negative pressure source 1 under consideration, which are assigned to different degrees or measures of wear. By comparing (using difference formation, quotient formation, etc.), for example, the time duration Δtₓ measured at the negative pressure source 1 under consideration or the determined angle αx as the value to be assessed with a corresponding reference value, a deviation of the value to be assessed from the corresponding reference value may be determined. This deviation may again be assigned to a measure of wear stored for this purpose in the storage device 5.

Thus, not only a comparison of the value to be assessed with the corresponding reference value may render possible stating a fact about the wear of the negative pressure source 1, but rather also the observation of only the value to be assessed (angle, time, etc.) and the reading out of a measure of wear assigned to its amount or value in the storage device 5. Said measure of wear may be stored, for example in or via a table, data records or the like. Reference values are not required here.

By using the control device or closed-loop control device 50, a corresponding qualitative and/or quantitative statement on the degree of wear or an alarm regarding same may be output via the output device 55.

Alternatively or in addition to Δt₁ or α1 as an example of the value to be assessed, any indication or information of or about the shape or profile (slope/gradient, curvature, etc.) which is suitable for describing the pressure value profile P1, P2 may be understood as a value to be assessed, and the value stored in this case as a reference value may correspond to the value thus determined.

Finally, it should be mentioned that already the comparison of the measured pressure value profile P1, P2 as such with a reference pressure profile stored in the storage device 5 as such may be carried out. A measure of wear may in turn be assigned to the result of this comparison. For example, an image comparison may be made between the measured pressure value profile P1, P2 on the one hand and the stored reference pressure profile on the other. If the graph of the pressure value profile deviates too much from the graph of the reference pressure profile, if their shapes differ too much, etc., a measure of wear may in turn be assigned to this deviation. In several embodiments therefore, the shape of the measured pressure value profile P1, P2 may already be understood as a value to be assessed.

FIG. 3 shows a quantitative assessment of the wear condition of a negative pressure source 1 in a further pressure-time-diagram as an optional result of an evaluation.

The two curves show, in an analogous manner to the representation in FIG. 2, the measured pressure value profiles P1, P2.

FIG. 3 further shows the lower control limit LL and an upper control limit UL. These are limit values or thresholds which in some embodiments regularly play a role in the intended use of the treatment apparatus 100, since negative pressure is built up again whenever the negative pressure prevailing in the vacuum line 3 exceeds the upper control limit UL, i.e. the prevailing negative pressure has relieved to this value and needs to be built up again. For this purpose, the negative pressure source 1 is prompted by the control device or closed-loop control device 50 to start as intended. When determining the present measure of wear, an orientation based on the lower control limit LL is therefore advisable or recommended for practical reasons. Other reference pressures or reference pressure courses may of course also be used for carrying out the present invention.

The time Δt₂ of the pressure value profile P2 which was needed or required for the building-up of the same negative pressure (here: around −650 mbar) in order to reach the lower control limit LL is longer than the time Δt₁ of the pressure value profile P1 which was required in order to reach the lower control limit LL. It is noted that the values Δt₁ and Δt₂ in the diagram of FIG. 2 stood for a period of time which elapsed between the beginning of the building-up of (negative) pressure and reaching a preset negative pressure, whereas in FIG. 3 they indicate the end of that period of time. The point in time at which the negative pressure source 1 began to build up the negative pressure is not specifically indicated in FIG. 3.

The results of the comparison or evaluation may be quantified using known methods, for example with an indication in percent related to a reference value being based on empirical values. In the example of FIG. 3, the pressure value profile P1 when or upon falling below the lower control limit LL, would result in a higher percentage for describing a first capacity, e.g. 84%, which is determined from parameters of the pressure value profile P1, than the percentage for describing a second capacity, e.g. 40%, determined from parameters of the pressure value profile P2 when or upon exceeding the lower control limit LL.

The percentage may represent, or be assigned to, a quantitative statement, for example about the capacity, the remaining service life and/or the like. Such numbers or associations may be based on empirical values. They may, for example, be provided in tabular form and/or stored in the storage device 5. A predetermined percentage may be a criterion for an alarm output or for triggering an alarm.

If no alarm is triggered, the value or amount of the value to be assessed or the degree of wear of the negative pressure source 1 being determined based on this value, may, prompted by the control device or closed-loop control device 50, be stored in the storage device 5 or in another storage device (not shown in the figures). Such storage may also take place in the event of an alarm.

It may be provided that a wear curve is calculated from several measured pressure value profiles Px, measured for one and the same negative pressure source 1 at different times, and stored, if applicable, for example in the storage device 5 mentioned herein. The wear curve may be stored such that it can be read out or displayed by a service technician.

An evaluation of such a wear curve or of different wear recording and/or wear evaluation may be carried out, e.g. automatically. Corresponding information may be sent, e.g. to a service technician.

FIG. 4 shows a flow diagram of further components of an embodiment of the medical treatment apparatus 100, here a blood treatment apparatus, connected to an extracorporeal blood circuit 300, which can be connected to the vascular system of the patient, not shown, for treatment using double-needle access, or by using e.g. an additional Y-connector (reference numeral Y), for treatment using single-needle access. The blood circuit 300 may be present, optionally in sections thereof, in or on a blood cassette, which is referred to herein as an example of a disposable.

Pumps, actuators and/or valves in the area of the blood circuit 300 are connected to the treatment apparatus 100 or to a control device 150 encompassed by treatment apparatus 100.

The blood circuit 300 includes (or is connected to) an arterial patient tube clamp 302 and an arterial connection needle of an arterial section or of an arterial patient line, blood withdrawal line or first line 301. The blood circuit 300 also includes (or is connected to) a venous patient tube clamp 306 and a venous connection needle of a venous section, a venous patient line, a blood return line or a second line 305.

A blood pump 101 is provided in or at the first line 301, a substitute fluid pump 111 is connected to a dialysis liquid inlet line 104 for conveying fresh dialysis liquid, which is filtered in a further filter stage (F2) (substitute fluid). A substitute fluid line 105 may be fluidically connected to the dialysis liquid inlet line 104. Using the substitute fluid pump 111, substitute fluid may be introduced by pre-dilution, via a pre-dilution valve 107, or by post-dilution, via a postdilution valve 109, via assigned lines 107a or 109a into line sections, for example into the arterial line section 301 or into the venous line section 305 (here between a blood chamber 303b of a blood filter 303 and a venous air separation chamber or venous blood chamber 329) of the blood circuit 300.

The blood filter 303 includes the blood chamber 303b connected to the arterial line section 301 and to the venous line section 305. A dialysis liquid chamber 303a of the blood filter 303 is connected to the dialysis liquid inlet line 104 leading to the dialysis liquid chamber 303a and to a dialysate outlet line 102, which guides dialysate, i.e. spent dialysis liquid, leading away from the dialysis liquid chamber 303a. Dialysis liquid chamber 303a and blood chamber 303b are separated from each other by a mostly semi-permeable membrane 303c. It represents the partition between the blood side with the extracorporeal blood circuit 300 and the machine side with the dialysis liquid circuit or dialysate circuit, which is shown in FIG. 4 to the left of the membrane 303c.

The dialysis liquid inlet line 104 is optionally in fluid communication with a compressor 175

The arrangement in FIG. 4 encompasses an optional detector 315 for detecting air and/or blood. The arrangement of FIG. 4 further encompasses one or two pressure sensors PS1 (upstream of the blood pump 101) and PS2 (downstream of the blood pump 101), the pressure sensors measure the pressure upstream of the blood filter 303 (“pre-hemofilter”) at the points shown in FIG. 4. Further pressure sensors may be provided, e.g. pressure sensor PS3 downstream of the venous blood chamber 329.

An optional single-needle chamber 317 is used in FIG. 1 as a buffer and/or compensating reservoir in a single-needle procedure in which the patient is connected to the extracorporeal blood circuit 300 using only one of the two blood lines 301, 305.

The arrangement of FIG. 4 also encompasses an optional detector 319 for detecting air bubbles and/or blood.

An addition site 325 for Heparin or another anticoagulant may optionally be provided.

Blood leaving the blood filter 303 flows through an optional venous blood chamber 329, which may include a de-aeration device 318 and may be in fluid communication with the pressure sensor PS3.

On the left in FIG. 4, a mixing device 160 is shown, which provides a predetermined mixture for the respective solution from the containers A (for A-concentrate via concentrate supply 166) and B (for B-concentrate via concentrate supply 168) for use by the treatment apparatus 100. The solution contains heated water, heated e.g. in a heating device 162 (on-line, e.g. as reverse osmosis water or from bags) from the water source 155.

A pump 171, which can be referred to as concentrate pump or sodium pump, is fluidically connected to the mixing device 163 and a source of sodium, for example the container B, and/or conveys out of it. An optional pump 173 can be seen which is assigned to the container B, such as for bicarbonate.

A negative pressure source for building-up negative pressure (or pressure) may, for example, be seen in the optional negative pressure source with the reference numeral 1 which is, non-limiting, herein also referred to as compressor, which is here exemplarily connected to the port 1000 and/or to the further port 1000′ via the vacuum line 3. Alternative pressure sources, in particular negative pressure sources, may be provided.

Furthermore, FIG. 4 shows a drainage line 153 for the effluent. The optional heat exchanger 157 and a first flow pump 159, which is suitable for degassing, complete the arrangement shown.

A further pressure sensor may be provided as PS4 downstream of the blood filter 303 on the water side, but preferably upstream of the ultrafiltration pump 131 in the dialysate outlet line 102 for measuring the filtrate pressure or membrane pressure of the blood filter 303. Additional, optional pressure measuring points P may also be provided.

The exemplary arrangement shown in FIG. 4 includes a control device or closed-loop control device 150. It may be in wired or wireless signal communication with any of the components mentioned herein—especially or in particular with the blood pump 101—for controlling or regulating the blood treatment apparatus 100.

By using the device for the on-line mixing of the dialysis liquid, a variation of its sodium content, being controlled by the control device or closed-loop control device 150, is possible within certain limits. For this purpose, in particular the measured values determined by the conductivity sensors 163a, 163b may be taken into account. Should an adjustment of the sodium content of the dialysis liquid (sodium concentration) or of the substitute fluid turn out to be necessary or desired, this can be done by adjusting the conveyance rate of the sodium pump 171.

In addition, the treatment apparatus 100 includes means for conveying fresh dialysis liquid and dialysate. A first valve may be provided between the first flow pump 159 and the blood filter 303, which first valve opens or closes the inflow towards the blood filter 303 at the inlet side. A second, optional pump or flow pump 169 which conveys dialysate towards and/or through the drainage line 153 is provided e.g. downstream of the blood filter 303. A second valve V25 may be provided between the blood filter 303 and the second flow pump 169, which second valve V25 opens or closes the outflow at the outlet side.

Furthermore, the treatment apparatus 100 optionally includes a device 161 for balancing the flow flowing into and out of the dialyzer 303 on the machine side. The device 161 for balancing is preferably arranged in a line section between the first flow pump 159 and the second flow pump 169.

The treatment apparatus 100 further includes means, such as the ultrafiltration pump 131, for the precise removal of a volume of liquid from the balanced circuit, as predetermined by the user and/or by the control device 150.

Sensors such as the optional conductivity sensors 163a, 163b may further be provided in order to determine the conductivity, which in some embodiments is temperature-compensated, as well as the liquid flow upstream and downstream of the dialyzer 303.

Temperature sensors 165a, 165b may be provided individually or in groups. Temperature values supplied by them may be used to determine a temperature-compensated conductivity. Furthermore, they may be provided in order to monitor the temperature of the dialysis liquid or of the dialysate.

Further flow pumps in addition or alternatively to e.g. the one with the reference numeral 169 may also be provided.

A number of optional valves is respectively denoted with V in FIG. 4.

The control device 150 determines in several embodiments the electrolyte balance and/or liquid balance based on the measured values from the aforementioned optional sensors.

Filters F1 and F2 can be connected in series.

Even when using non-sterile water, the filter F1 exemplarily serves herein to generate sufficiently pure dialysis liquid by the mixing device 160, which then flows through the blood filter 303, e.g. using the countercurrent principle.

The filter F2 exemplarily serves here to generate sterile or sufficiently filtered substitute fluid from the sufficiently pure dialysis liquid leaving the first filter F1, by filtering e.g. pyrogenic substances. This substitute fluid may then be safely added to the extracorporeally flowing blood of the patient and thus ultimately to the patient's body.

The treatment apparatus 100 is optionally shown in FIG. 4 as an apparatus for hemo(dia)filtration. However, hemodialysis apparatuses are also covered by the present disclosure, although not specifically represented in a figure.

The present disclosure is not limited to the embodiment described above; this only serves for illustration.

The arrows shown in FIG. 4 generally indicate the flow direction respectively.

As valves, in particular valves V24 in the line 104 upstream of the blood filter 303, the valve V25 in the line 102 downstream of the blood filter 303 and the V33, V44, V43, V42, V45 may be provided at the sites which are indicated or shown.

### LIST OF REFERENCE NUMERALS

1 negative pressure source; e.g. compressor
3 vacuum line
5 storage device
7 reading device
9 determining device
11 evaluation device
50 control device or closed-loop control device
55 output device
100 medical treatment device
101 blood pump
102 dialysate outlet line
104 dialysis liquid inlet line
105 substitute fluid line
107 pre-dilution valve
107a line corresponding to or assigned to the pre-dilution valve
109 post-dilution valve
109a line corresponding to or assigned to the post-dilution valve
111 substitute fluid line
131 ultrafiltration pump
150 control device
153 drainage line or discharge line
155 water source
157 heating device; heat exchanger
159 first flow pump
160 mixing device
161 device for the balancing
162 heating device
163a conductive sensor
163b conductive sensor
165a temperature sensor
165b temperature sensor
166 concentrate supply
168 concentrate supply
169 second flow pump
171 pump, sodium pump
173 pump, bicarbonate pump
175 compressor
300 extracorporeal blood circuit
301 first line (arterial line section)
302 (first) tube clamp
303 blood filter or dialyzer
303a dialysis liquid chamber
303b blood chamber
303c semi-permeable membrane
305 second line (venous line section)
306 (second) tube clamp
315 detector
317 single-needle chamber
318 de-aeration device
319 detector
325 addition site for anticoagulant
329 venous blood chamber (optional)
1000 port
1000′ port
F1 filter
F2 filter
F3 filter for sterile air
F4 filter for sterile air
A container
B container
DS pressure-measuring device
LL lower control limit
P1 measured pressure value profile
P2 measured pressure value profile
P pressure measuring sites
PS1 arterial pressure sensor (optional)
PS2 arterial pressure sensor (optional)
PS3 pressure sensor (optional)
PS4 pressure sensor for measuring the filter pressure (optional)
S03 pressure sensor
S07 pressure sensor
Δt₁, Δt₂, . . . value to be assessed
α1, a2, . . . value to be assessed
UL upper control limit
V valves
V24 Valve
V25 Valve
V31 Valve
V32 Valve
V33 Valve
V42 Valve
V43 Valve
V44 Valve
V45 Valve
Y Y-connector

1-12. (canceled)

13. A medical treatment apparatus comprising, or connected to:
a vacuum line;
a negative pressure source, which is in fluid communication or in conveying communication with the vacuum line, for building up a negative pressure in the vacuum line;
a pressure measuring device for measuring pressure values of the negative pressure while obtaining a measured pressure value profile;
a determining device configured for determining at least one value to be assessed from the measured pressure value profile;
an output device; and
a storage device, in which there is stored:
at least one reference pressure profile or at least one reference value, being related to a reference pressure profile, for the at least one value to be assessed, and/or
at least one measure assigned to the at least one value to be assessed, wherein the at least one measure indicates a present or an existing wear of the negative pressure source;
a reading device configured to read out, from the storage device, the stored reference pressure profile, the at least one reference value, or the at least one measure; and
a control device or closed-loop control device configured to prompt steps including:
building up or amending the negative pressure in the vacuum line by using the negative pressure source;
measuring the negative pressure prevailing in the vacuum line by using the pressure measuring device, while obtaining a measured pressure value profile;
determining the at least one value to be assessed from the measured pressure value profile by using the determining device;
reading out from the storage device using the reading device:
the at least one reference pressure profile or the at least one reference value corresponding to the at least one value to be assessed; or
the at least one measure assigned to the at least one value to be assessed; and
outputting, using the output device, a result determined by the control device or closed-loop control device of a comparison between the at least one reference pressure profile or the at least one reference value and the at least one value to be assessed, or the at least one measure assigned to the at least one value to be assessed.

14. The medical treatment apparatus according to claim 13, further comprising or connected to:
an evaluation device configured to assign a measure of wear to the result of the comparison;
wherein the control device or closed-loop control device is further configured to assign, using the evaluation device, the measure of wear, and is configured to output the measure of wear using the output device.

15. The medical treatment apparatus according to claim 14, wherein the control device or closed-loop control device is further configured to determine whether the at least one value to be assessed, the result of the comparison, and/or the measure of wear is within predetermined limits, exceeds or falls below a limit value or threshold, exceeds a minimum value and/or does not exceed a maximum value.

16. The medical treatment apparatus according to claim 13, wherein the at least one reference value and/or the at least one value to be assessed is a time value or a mathematical parameter.

17. The medical treatment apparatus according to claim 16, wherein the at least one reference value and/or the at least one value to be assessed is an angle.

18. The medical treatment apparatus according to claim 13, wherein the control device or closed-loop control device is configured to prompt an outputting, using the output device, of an acoustic and/or optical alarm should the result or the at least one measure assigned to the at least one value to be assessed assume values or characteristics defined in advance as impermissible.

19. The medical treatment apparatus according to claim 18, wherein the result or the at least one measure assigned to the at least one value to be assessed assume the values or the characteristics defined in advance as impermissible upon leaving predetermined limits.

20. The medical treatment apparatus according to claim 18, wherein the result or the at least one measure assigned to the at least one value to be assessed assume the values or the characteristics defined in advance as impermissible upon falling below a lower limit value or exceeding a upper limit value, upon leaving a permissible value range, or upon exceeding a predetermined amount.

21. The medical treatment apparatus according to claim 13, wherein the control device or closed-loop control device is configured to cause a storing of the result or of the at least one measure assigned to the at least one value to be assessed by using said storage device.

22. The medical treatment apparatus according to claim 21, wherein the control device or closed-loop control device is configured to carry out or prompt a determination of a wear profile for the negative pressure source from the stored results or the at least one measure assigned to the at least one value to be assessed.

23. The medical treatment apparatus according to claim 21, wherein the determination of the wear profile is a calculation of the wear profile.

24. The medical treatment apparatus according to claim 13, embodied as a blood treatment apparatus, as a hemodialysis apparatus, as a hemofiltration apparatus, as a hemodiafiltration apparatus, or as an apparatus for executing a separation procedure.

25. A control device or closed-loop control device configured to prompt:
building up or amending a negative pressure in a vacuum line by using a negative pressure source;
measuring the negative pressure in the vacuum line by using a pressure measuring device, while obtaining a measured pressure value profile;
determining at least one value to be assessed from the measured pressure value profile by using a determining device;
reading out from a storage device using a reading device:
at least one reference pressure profile or at least one reference value corresponding to the at least one value to be assessed; or
at least one measure assigned to the at least one value to be assessed; and
outputting, using an output device, a result determined by the control device or closed-loop control device of a comparison between the at least one reference pressure profile or the at least one reference value and the at least one value to be assessed, or the at least one measure assigned to the at least one value to be assessed.

26. A digital storage medium with electronically readable control signals, configured for configuring a control device or closed-loop control device into a control device or closed-loop control device according to claim 25.

27. The digital storage medium according to claim 26, in the form of a floppy disk, CD, DVD, or an EPROM.

28. A computer program product with a program code stored on a machine-readable carrier for configuring a control device or closed-loop control device into a control device or closed-loop control device according to claim 25.

29. A computer program with a program code for configuring a control device or closed-loop control device into a control device or closed-loop control device according to claim 25.
